(19) United States
(12) Patent Application Publication
(10) Pub. No.: US 2014/0330254 A1
(43) Pub. Date: Nov. 6, 2014

(54) CHANNEL SEPARATION DEVICE AND RELATED METHOD THEREOF

(71) Applicant: UNIVERSITY OF VIRGINIA PATENT FOUNDATION, Charlottesville, VA (US)

(72) Inventors: Laura H. Rosenberger, Charlottesville, VA (US); Robert G. Sawyer, Charlottesville, VA (US); Christopher M. Watson, West Columbia, SC (US)

(73) Assignee: UNIVERSITY OF VIRGINIA PATENT FOUNDATION, Charlottesville, VA (US)

(21) Appl. No.: 14/365,272

(22) PCT Filed: Dec. 14, 2012
(86) PCT No.: PCT/US2012/069793

§ 371 Date: Jun. 13, 2014

Related U.S. Application Data
(60) Provisional application No. 61/576,775, filed on Dec. 16, 2011.
(60) Provisional application No. 61/587,580, filed on Jan. 17, 2012.

(51) Int. Cl. A61M 39/10; A61J 15/00

(52) U.S. Cl. CPC A61M 39/1011; A61J 15/0015; A61J 15/0026
USPC 604/535

(57) ABSTRACT

A device, and method for using and manufacturing that device, to prevent accidental disruption of a channel placed inside a subject. The device is placed external to the patient, in-line with the channel, and it is designed to separate when a desired tractive force is applied to a portion of the channel that is outside the subject.

### CROSS REFERENCE TO RELATED APPLICATIONS

The present application claims priority under 35 U.S.C. §119(e) from U.S. Provisional Application Ser. No. 61/576,775, filed Dec. 16, 2011, entitled “Coupling Device and Related Method” and Ser. No. 61/587,580, filed Jan. 17, 2012, entitled “Coupling Device and Related Method;” the disclosures of which are hereby incorporated by reference herein in their entirety.

### FIELD OF THE INVENTION

The present invention relates generally to the field of medical devices. More specifically, the invention is in the subfield of tubing, channels, drains, catheters, and ports connected to a subject patient.

### BACKGROUND OF THE INVENTION

The percutaneous endoscopic gastrostomy, or the PEG, as it is now most commonly known, was first introduced in 1980 by Gauderer, Ponsky, and Izant. Currently, more than 215,000 PEGs are placed annually [See 1, Gauderer, 2001]. Due to the design of the PEG, a major complication is “premature removal” or accidental dislodgement, from which significant morbidity and mortality may occur [See 4, Shapiro, 1996]. Secondary complications occur with premature removal, as the gastrocutaneous fistula tract has not fully matured, allowing the stomach to separate from the anterior abdominal wall and the open gastrostomy to leak gastric contents. Accidental dislodgement rates are well published and have been reported up to 12.8% when followed longitudinally for the lifetime of the originally placed PEG. [See 9, Chowdhury]

While early dislodgements can be clinically devastating, late dislodgements after gastrocutanous fistula maturation are less detrimental, however, they may require expensive emergency department visits, surgical consultations, replacement tubes, and radiographic confirmation of position [See 10, Rosenberger].

The mechanism that causes disruption of the PEG, may also cause disruption of any channel that is positioned inside a subject. Catheters, drains, and various other tubes are all subject to disruption when tractive force is applied to the portion of those tubes that are external to the subject.

Therefore, there is need in the art for a device which reduces the disruption of channels placed inside a subject.

### SUMMARY OF THE INVENTION

An aspect of an embodiment of the present invention provides an assembly device for use in a medical environment to be used with a first channel and a second channel, wherein the first channel has an internal portion located at an intended position inside a subject. The device may comprise: a first connector comprising a first interface member and a channel communication section; a second connector comprising a second interface member and a channel communication section; said first interface member of said first connector and said second interface member of said second connector configured to join together to form a coupling configured to allow fluid to flow between the first channel and the second channel, said coupling having a decoupling force; wherein said coupling is joined to the first channel and the second channel at said communication section of said first connector and said communication section of said second connector; and wherein said decoupling force is the force required to separate said first connector and the second connector from one another to allow the internal portion to maintain its intended position in the subject.

An aspect of an embodiment of the present invention provides an assembly device for use in a medical environment to be used with a first channel and a second channel, wherein the first channel has an internal portion located at an intended position inside a subject. The device may comprise: a first connector; a second connector; said first connector and said second connector configured to join together to form a coupling configured to allow fluid to flow between the first channel and the second channel, said coupling having a decoupling force; wherein said coupling is joined between the first channel and the second channel; and wherein said decoupling force is the force required to separate said first connector and the second connector from one another to allow the internal portion to maintain its intended position in the subject.

An aspect of an embodiment of the present invention provides a system for use in a medical environment. The system may comprise: a first channel; a second channel, wherein the first channel has an internal portion located at an intended position inside a subject; and an assembly device. The assembly device may comprise: a first connector comprising a first interface member and a channel communication section; a second connector comprising a second interface member and a channel communication section; said first interface member of said first connector and said second interface member of said second connector configured to join together to form a coupling configured to allow fluid to flow between said first channel and said second channel, said coupling having a decoupling force; wherein said coupling is joined to said first channel and said second channel at said communication section of said first connector and said communication section of said second connector; and wherein said decoupling force is the force required to separate said first connector and the second connector from one another to allow the internal portion to maintain its intended position in the subject.

An aspect of an embodiment of the present invention provides a system for use in a medical environment, wherein said system comprises a first channel; a second channel, wherein the first channel has an internal portion located at an intended position inside a subject; and an assembly device. The assembly device may comprise: a first connector; a second connector; said first connector and said second connector configured to join together to form a coupling configured to allow fluid to flow between said first channel and said second channel, said coupling having a decoupling force; wherein said coupling is joined between said first channel and said second channel; and wherein said decoupling force is the force required to separate said first connector and the second connector from one another to allow the internal portion to maintain its intended position in the subject.

An aspect of an embodiment of the present invention provides a method used in a medical environment to be used with a first channel and a second channel, wherein said first channel has an internal portion located at an intended position inside the subject. The method may comprise: providing a first connector; providing a second connector; joining said first connector and said second connector to form a coupling to allow fluid to flow between the first channel and the second channel, said coupling having a decoupling force; wherein said coupling is joined to the first channel and the second channel; and wherein said decoupling force is the force required to separate said first connector and said second connector from one another to allow the internal portion to maintain its intended position.

An aspect of an embodiment of the present invention provides a method used in a medical environment. The method may comprise: providing a first channel; providing a second channel, wherein said first channel has an internal portion located at an intended position inside the subject; providing a first connector; providing a second connector; joining said first connector and said second connector to form a coupling to allow fluid to flow between said first channel and said second channel, said coupling having a decoupling force; wherein said coupling is joined to said first channel and said second channel; and wherein said decoupling force is the force required to separate said first connector and said second connector from one another to allow the internal portion to maintain its intended position.

These and other advantages and features of the invention disclosed herein, will be made more apparent from the description, drawings and claims that follow.

### DESCRIPTION OF THE DRAWINGS

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 provides a schematic illustration of the assembly device in use with a subject.

FIGS. 2(A) and 2(B) provide schematic illustrations of the assembly device in use with a subject. FIGS. 2(A) and 2(B) also illustrate an embodiment of the first interface member on the first connector and an embodiment of the second interface member on the second connector. FIG. 2(B) further illustrate an embodiment of the protrusions of the first interface member and the protrusion retention recesses of the second interface member.

FIGS. 3(A)-(D) provide sectional views of several specific embodiments of the first connector having different embodiments of the channel communication section. It should be appreciated that these embodiments of the channel communication section are equally applicable to the second connector.

FIG. 4 provides a sectional view of an embodiment of the assembly device in use with a subject.

FIG. 5(A) provides an elevation view of an embodiment of the first connector. FIG. 5(B) provides a sectional view designated as A-A for the embodiment shown in FIG. 5(A).

FIG. 5(C) provides an enlarged partial view designated as Detail B of the sectional view shown in FIG. 5(B).

FIG. 6(A) provides an elevation view of an embodiment of the second connector.

FIG. 6(B) provides a sectional view designated as B-B for the embodiment shown in FIG. 6(A).

FIG. 6(C) provides an enlarged partial view designated as Detail A of the sectional view shown in FIG. 6(B).

FIG. 7(A) provides an elevation view of an embodiment of the coupling formed by the joining of the first connector shown in FIG. 5(A) and the second connector show in FIG. 6(A).

FIG. 7(B) provides a sectional view designated as C-C for the embodiment shown in FIG. 7(A).

FIG. 7(C) provides an enlarged partial view designated as Detail D of the sectional view shown in FIG. 7(B).

FIGS. 8(A)-8(B) provide a perspective view of an embodiment of the coupling in a disconnected position and connected position, respectively.

FIG. 9(A) provides an illustration of an elevation schematic view of a PEG tube. FIGS. 9(B)-(C) provides an elevation schematic view of an embodiment of the coupling in a disconnected position and connected position, respectively.

FIG. 10 graphically illustrates the force (kg) of external traction for removal of PEG tubes through abdominal wall versus the thickness (cm) of the abdominal wall.

FIG. 11 graphically illustrates the force of external traction (kg) versus “age” (days) of the PEG tube.

FIG. 12 graphically illustrates the Standard (Gaussian) distribution with mean (g) and standard deviations (a) used for determining the preferred break force.

FIG. 13 graphically illustrates the Kaplan-Meier analysis for current prospective cohort (Group 2) versus historic cohort (Group 1).

### DETAILED DESCRIPTION OF EXEMPLARY EMBODIMENTS OF THE INVENTION

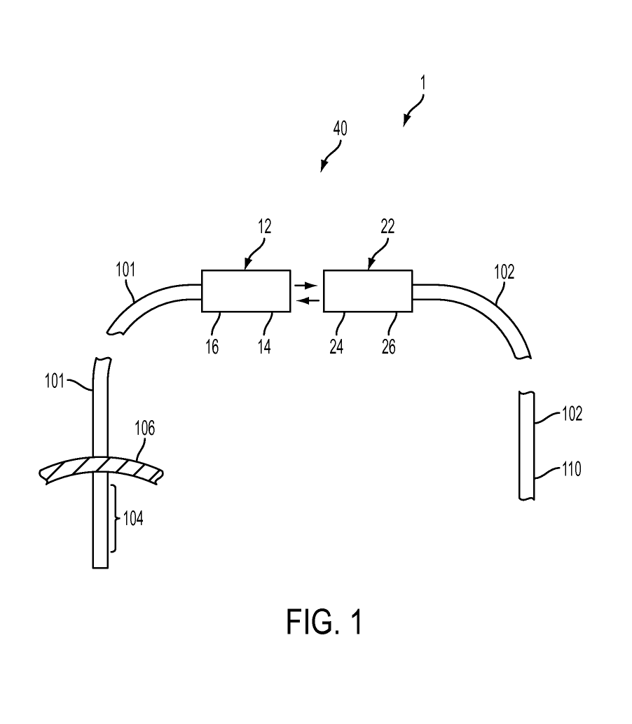

FIG. 1, provides a schematic illustration of the assembly device 1 in use with a subject. The device 1 is used in a medical environment and is to be used with a channel such as the first channel 101 and second channel 102 shown, and wherein the channel has an internal portion 104 that is located at an intended position inside a subject 106. The device 1 includes a first connector 12 having a first interface member 14 and a channel communication section 16 and a second connector 22 having a second interface member 24 and a channel communication section 26. The first interface member 14 of the first connector 12 and the second interface member 24 of the second connector 22 are configured to join together in the general directions of the arrows, to form a coupling 40 that is configured to allow fluid (or other material or gas) to flow between the first channel 101 and the second channel 102. The coupling 40 exhibits a desired or predetermined decoupling force. In an embodiment, the coupling 40 is joined to the first channel 101 and the second channel 102 at a communication section 16 of the first connector 12 and a communication section 26 of the second connector 22 in such manner so as to exhibit the decoupling force whereby the decoupling force is the force required to separate the first connector 12 and the second connector 22 from one another to allow the internal portion 104 to maintain its intended position in the subject 106. Moreover, the separated first connector 12 and second connector 22 is able to remain joined to their respective channels.

It should be appreciated that the first connector and second connector may be interchanged with one another respective to the first channel and second channel.

Still referring to the assembly device 1 of FIG. 1, it should be appreciated that the first connector 12 and the second connector 22 forming the coupling 40 may be achieved by a number of designs and approaches. Some examples include, but are not limited to, male to female friction connectors, magnetic connectors, connectors that use adhesive, friction connection, any ridge/valley connection, snap/click connection, any O-ring connection, and screw/twist type mechanisms.

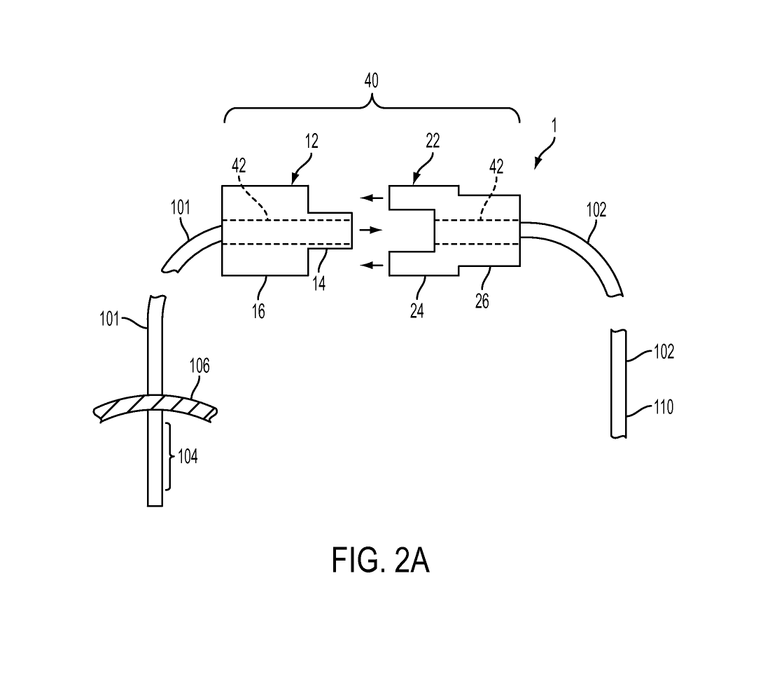

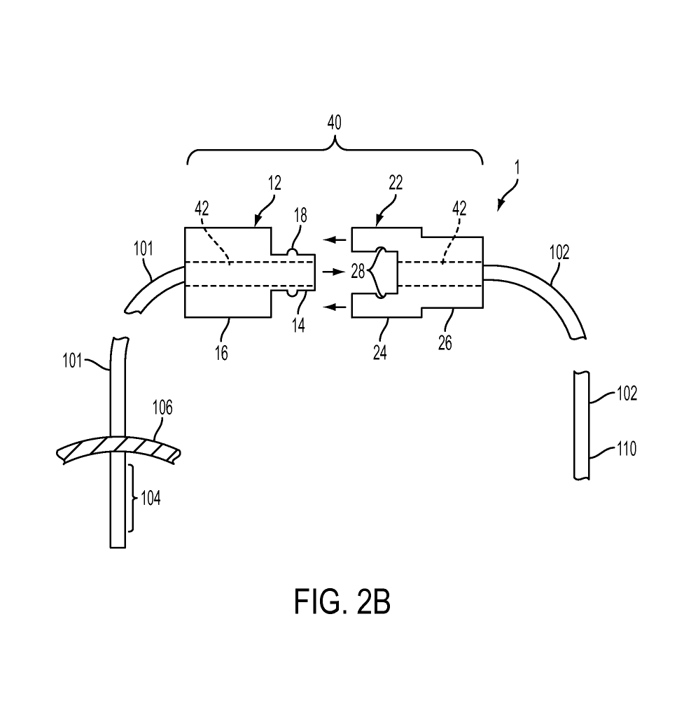

For instance, referring to the device of assembly of FIG. 2(A), which provides schematic illustrations of the assembly device 1 in use with a subject 106, the first connector 12 may include a male connector whereby the first interface member 14 provides an insert section. Similarly, the second connector 22 may include a female connector whereby the second interface member 24 provides a receiving section. As a result, the receiving section of the female connector may receive the insert section of the male connector so as to join together the two connectors to form the coupling 40.

Referring to FIG. 1 and FIGS. 2(A) and 2(B), the channel (e.g., first channel 101 and second channel 102) may include at least one of the following: a tube, a conduit, a port or any combination thereof. By way of example, in an embodiment the tube or the like may be connected to the channel communication section 16 of the first coupling 12, and that tube or the like may be connected to a port (not shown) that is within and/or adjacent to the internal portion 104 inside the subject 106. As such, the tube and the port would be connected to comprise the first channel 101. Next, in an embodiment the distal end 110 of the second channel 102 may be attached to a biomedical device such as a feeding apparatus or drainage collection vessel; however, it should be noted that the invention is not limiting in this regard and the terminus of the second channel is determined by any number of medical uses.

Again referring to FIG. 1 and FIGS. 2(A) and 2(B), in an embodiment the first channel 101 and second channel 102 may comprise a PEG tube, such that once fully assembled, the coupling 40 is located between the subject 106 and the distal end 110 of the second channel 102.

Still referring to FIG. 1 and FIGS. 2(A) and 2(B), the coupling 40 may have a decoupling force between 0.5 kilogram-force and 2.5 kilograms-force. The coupling 40 will separate when an appropriately oriented axial force is applied to the first connector 12 and second connector 22. The force required to decouple the coupling 40 is designed to be less than the force required to disrupt the position of the internal portion 104 of the first channel 101 that is inside the subject 106. In this way, the second channel 102 may be pulled either inadvertently or purposefully and the coupling 40 will separate before the force required to disrupt the portion of the first channel 101 inside the subject 106 is reached.

Still referring to FIG. 1 and FIGS. 2(A) and 2(B), the coupling 40 may have a decoupling force between 1.1 kilograms-force and 1.3 kilograms-force. The coupling 40 will separate when an appropriately oriented axial force is applied to the first connector 12 and second connector 24. The force required to decouple the coupling 40 is designed to be less than the force required to disrupt the position of the internal portion 104 of the first channel 101 that is inside the subject 106. In this way, the second channel 102 may be pulled either inadvertently or purposefully and the coupling 40 will separate before the force required to disrupt the portion of the first channel 101 inside the subject 106 is reached.

It should be appreciated that the decoupling force may be increased or decreased so as to comply with operative, structural and anatomical demands associated with design and practice of the various embodiments disclosed herein.

Referring now to FIG. 2(B), providing schematic illustrations of the assembly device 1 in use with a subject 106, illustrates an embodiment including the protrusions 18 of the first interface member 14 and the protrusion retention recesses 28 of the second interface member 24. When the first interface member 14 is inserted into the second interface member 24, the circumferentially oriented protrusions 18 on the first interface member 14 causes the second interface member 24 to deform radially outward. This outward deformation continues to increase as the first interface member 14 is inserted farther into the second interface member 24. When the first interface member 14 reaches its intended position inside the second connector 22, the circumferentially oriented protrusions 18 on the first interface member 14 come to rest inside the protrusion retention recess or recesses 28 of the second interface member 24. This causes the second interface member 24 to return to its original, non-deformed, shape and the coupling 40 is created. When the coupling 40 is created, a leak-inhibiting seal is created at the faying surfaces of the connection. The faying surfaces are all of the surfaces where the first connector 12 and the second connector 22 are in contact. It is well understood in the art that the dimensions, tolerances, and materials of the first and second connector affect the leak-inhibiting properties of the coupling 40.

Still referring to FIG. 2(B), the circumferentially oriented protrusions 18 may take many forms, including, but in no way limited to, a single continuous circumferential rib, a segmented circumferential rib, or multiple combinations of either form. The protrusion retention recess or recesses 28 are designed to accept the protrusions on the male connector insert section when the two connectors are joined. In this way, if there are multiple continuous circumferential ribs on the first interface member 14, there shall be multiple protrusion retention recesses on the second interface member 24. It should be appreciated that a single protrusion retention recess 28 may be adequate to accept a segmented circumferential rib, in this way, the radial orientation of the first connector 12 and the second connector 24 may be of no consequence when the connector are joined together to form a coupling. It is appreciated that a continuous circumferential rib may provide better leak-inhibiting characteristics than a segmented circumferential rib.

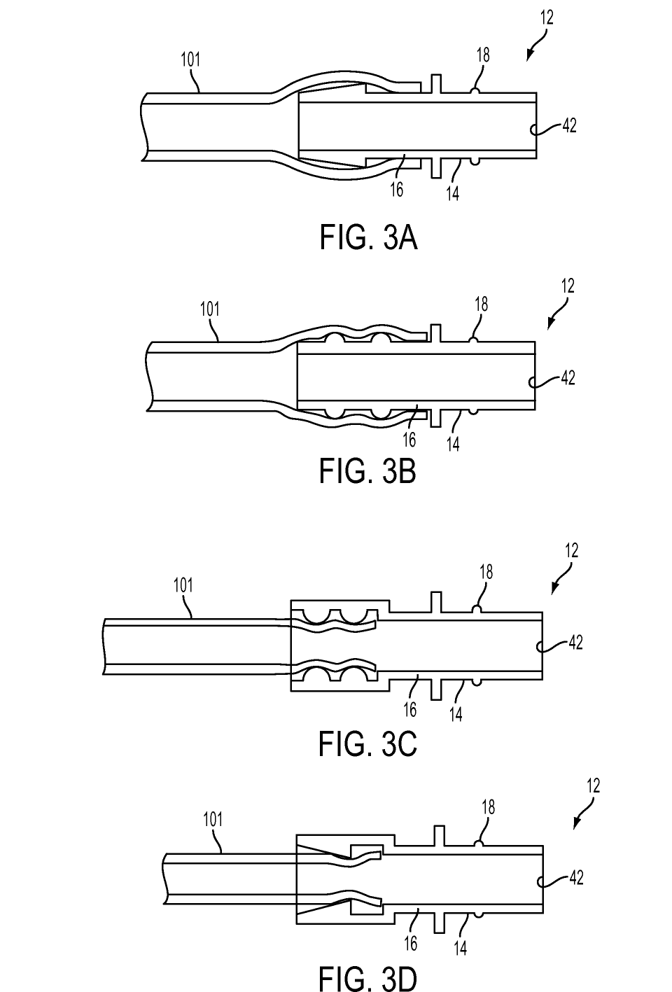

Referring now to FIGS. 3(A)-(D), which provide sectional views of several specific embodiments of the channel communication section 16 typical on the first connector 12. The channel communication section 16 of the first connector 12 (and channel communication section 26 the second connector 22 although not shown in FIG. 3) can be arranged in various forms known in the art. The first channel 101 (or second channel 102, although not shown in FIG. 3) may take the form of a tube, conduit, port, or other similar apparatus. As shown in FIGS. 1 and 2, in an embodiment, a first tube 101 will be attached to the first connector 12 and a second tube 102 will be attached to the second connector 22 at their respective channel communication sections, 16 and 26). FIGS. 3(A)-(D) illustrate various, but not exhaustive, means to attach a first channel 101 to the channel communication section 16 of the first connector 12. It should be appreciated that the same channel communication configurations shown on the first connector 12 also will apply equally to the second connector 22. In FIGS. 3(A) and 3(B), a first channel 101 is extended radially outward as the connector 12 is inserted into the first channel 101. The first channel 101 expands to accept the connector 12 and is held in place by a friction force. This friction force is enhanced by an appendage 17, such as the barb-like extension shown in FIG. 3(A) and the two circumferentially oriented ribs extensions shown in FIG. 3(B). FIGS. 3(C) and 3(D) show configurations of the channel insert section 16 where the first channel 101 is compressed radially inward as the connector 12 is pushed over the first channel 101. In FIG. 3(C), the channel 101 is held in place by a friction force generated by the appendage 17, such as circumferentially oriented ribs on the inside of the connector 12. In FIG. 3(D), the channel 101 is held in place by a friction force generated by the appendage 17, such as the barb-like extension on the inside of the connector 12. Various combinations or means for connecting the channel 101 with the connector 12 shown in FIGS. 3(A)-(D) may be used to obtain the desired strength or means of connection and it is understood that there are many other means commonly used in the art to attach a channel, tube, conduit, or port to a connector (12 or 22). Some other examples may include, but are not limited to, other type of male to female friction connectors, magnetic connectors, connectors that use adhesive, friction connection, any ridge/valley connection, snap/click connection, any O-ring connection, and screw/twist type mechanisms.

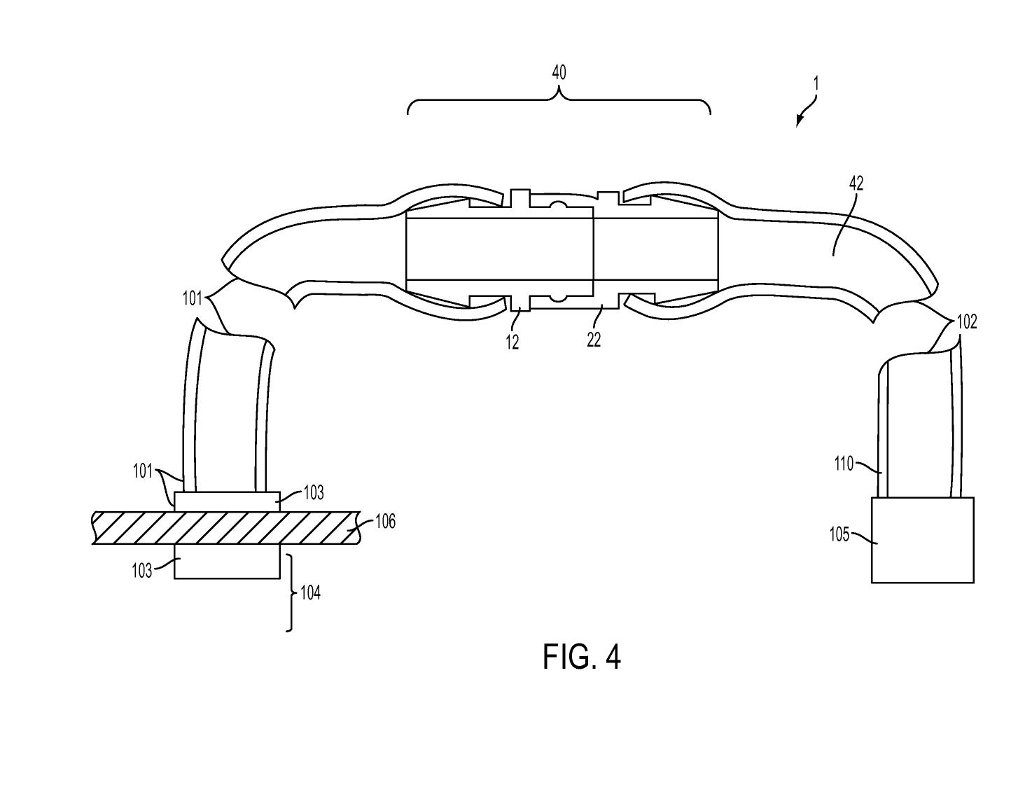

Referring now to FIG. 4, FIG. 4 provides schematic illustrations of the assembly device 1 in use with a subject 106. It is shown that the first channel 101 may be a tube that is connected to the channel communication section 16 of the first coupling 12, and that tube may be connected to or be integral with a port 103 that has an internal portion 104 inside the subject 106. Both the tube 101 and the port 103 connected together are may constitute portions of the first channel 101. The distal end 110 of the second channel 102 is shown to be attached to a device or instrument 105 (biomedical device), such as a feeding apparatus or drainage collection vessel; however, it should be noted that the invention is not limiting in this regard and the terminus of the second channel 102 may be determined by any number of medical uses.

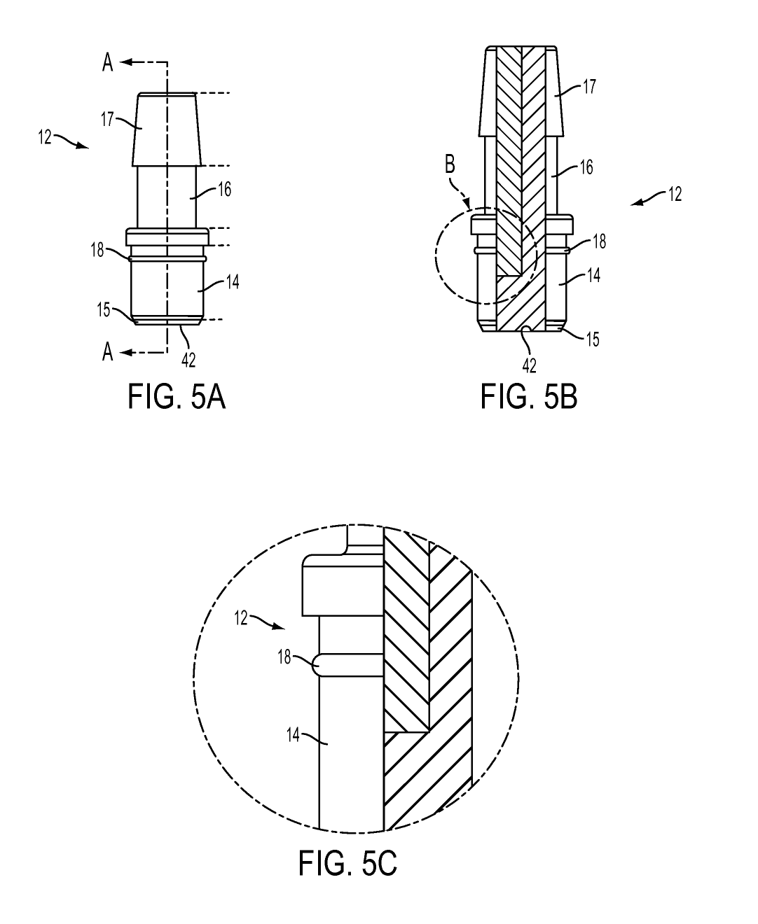

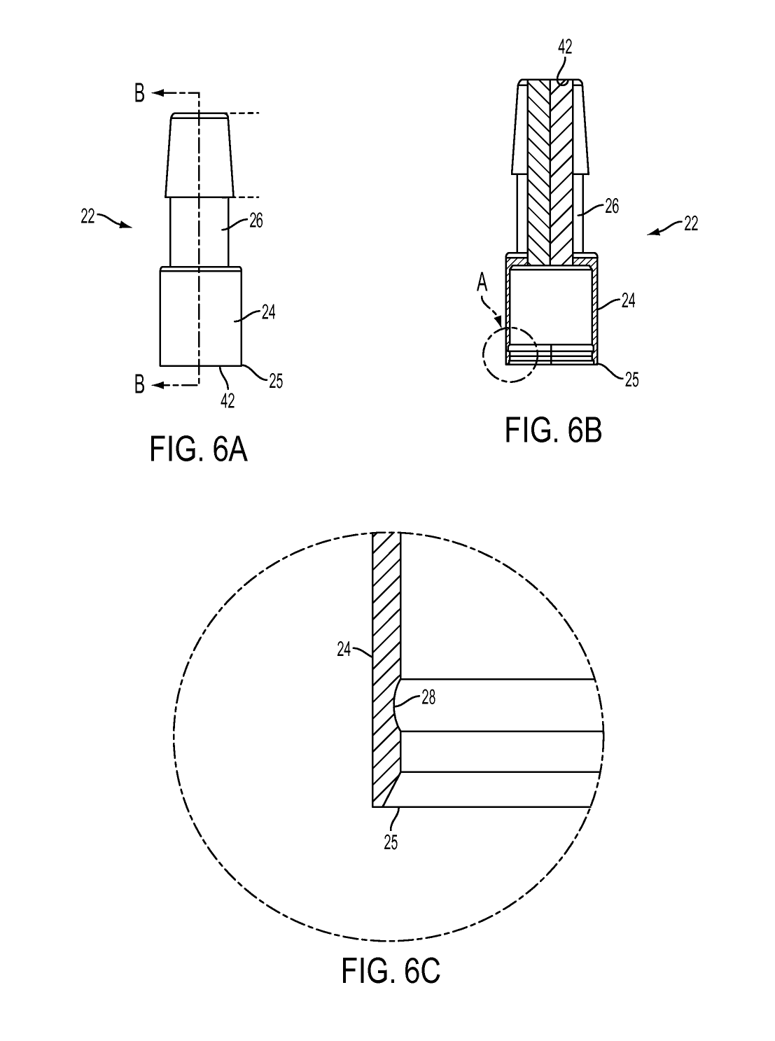

Referring now to FIGS. 5(A)-(C), which provides assorted views of an embodiment of a first connector 12, FIG. 5(A) shows an elevated schematic of a first connector 12. In this instance, the first interface member 14 is a male type connector with a first edge 15. The first edge 15 may include a chamber or radius to facilitate entry into the female type second interface member 24 shown in FIG. 6(A). FIG. 5(A) also illustrates a circumferentially oriented protrusion 18 in the form of a single continuous rib on the first interface member 14. FIG. 5(A) further illustrates a male type channel communication section. This type of channel communication section is an exemplary (non-limiting) embodiment for connection to typical PEG tubes.

FIG. 5(B) provides a sectional view designated as A-A for the embodiment shown in 5(A), and as such shows the fluid flow path 42.

FIG. 5(C) provides an enlarged partial view designated as Detail B of the sectional view shown in FIG. 5(B). FIG. 5(C) illustrates a cross sectional view of the circumferentially oriented protrusion 18, in this case embodied as a single continuous rib.

Referring now to FIGS. 6(A)-(C), which provide assorted views of an embodiment of a second connector 22, FIG. 6(A) shows an elevated schematic of a second connector 22. In this instance, the second interface member 24 is a female type connector with a first edge 25. The first edge 25 has a chamber or radius to facilitate entry of the male type first interface member 14 shown in FIG. 5(A). FIG. 6(A) further illustrates a male type channel communication section. This type of channel communication section is an exemplary (non-limiting) embodiment for connection to typical PEG tubes.

FIG. 6(B) provides a sectional view designated as B-B for the embodiment shown in 6(A), and as such shows the fluid flow path 42.

FIG. 6(C) provides an enlarged partial view designated as Detail A of the sectional view shown in FIG. 6(B). FIG. 6(C) illustrates a cross sectional view of the protrusion retention recess 28 as shown to accept the circumferentially oriented protrusion 18 of FIG. 5(A). FIG. 6(C) also illustrates a chamber on the first edge 25.

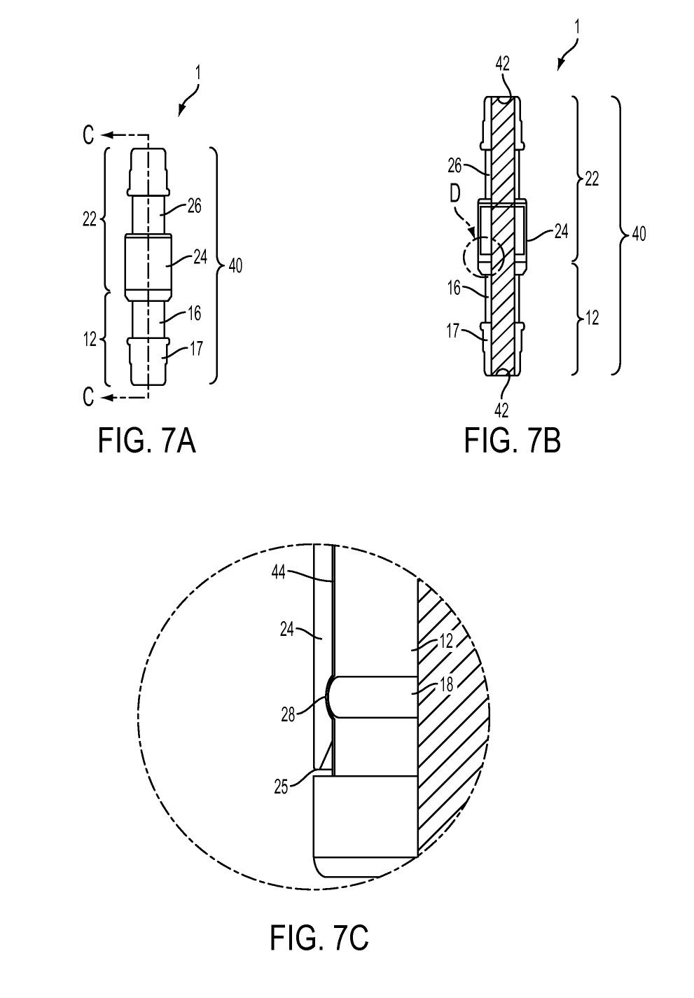

Referring now to FIG. 7(A), FIG. 7(A) provides and elevation view of an embodiment of the coupling 40 formed by the joining of the first connector 12 shown in FIG. 5(A) and the second connector 22 show in FIG. 6(A).

FIG. 7(B) provides a sectional view designated as C-C for the embodiment shown in FIG. 7(A). FIG. 7(B) illustrates the embodiment of the coupling 40 formed by the joining of the first connector 12 shown in FIG. 5(A) and the second connector 22 show in FIG. 6(A). The fluid flow path 42 through the coupling is identified.

FIG. 7(C) provides an enlarged partial view designated as Detail D of the sectional view shown in FIG. 7(B). In this partial view it is shown how a cross section of the circumferentially oriented protrusion 18 and the protrusion retention recess 28 communicate while the first connector 12 and the second connector 22 are joined to form a coupling 40. The faying surface 44 of the coupling is also shown.

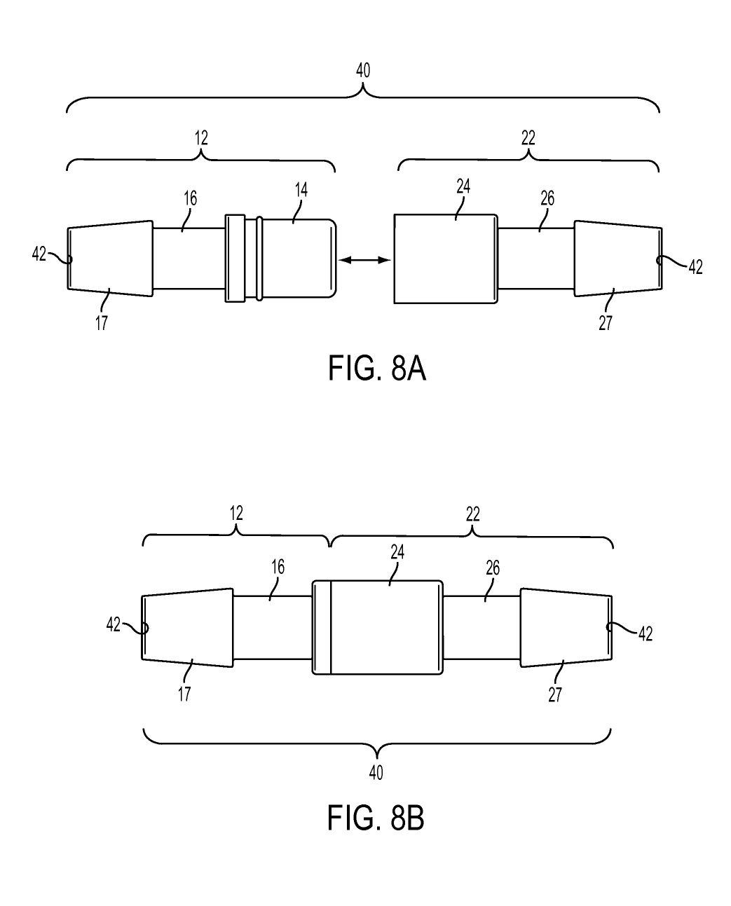

Referring now to FIG. 8, FIGS. 8(A)-8(B) provide a perspective view of an embodiment of the coupling 40 in a disconnected position and connected position, respectively. FIG. 8(A) provides a perspective view of an embodiment of the coupling 40 in a disconnected position, which includes a first connector 12 that is disconnected from a second connector 22. As shown, the channel communication section 16 has a barb-like appendage 17 to facilitate communication with a channel (not shown). Similarly, the channel communication section 26 has a barb-like appendage 27 to facilitate with a channel (not shown). The first connector 12 and the second connector 22 are configured to provide a fluid flow path 42 there through. FIG. 8(B) provides a perspective view of an embodiment of the coupling 40 of the coupling shown in FIG. 8(A) whereby the first connector 12 is joined to the second connector 22 to form the coupling 40. The coupling 40 is configured to provide for the fluid flow path 42 to extend through the coupling 40 to allow fluid to flow from the first connector 12 to the second connector 22. As shown, the channel communication section 16 has a barb-like appendage 17 to facilitate communication with a channel (not shown). Similarly, the channel communication section 26 has a barb-like appendage 27 to facilitate communication with a channel (not shown).

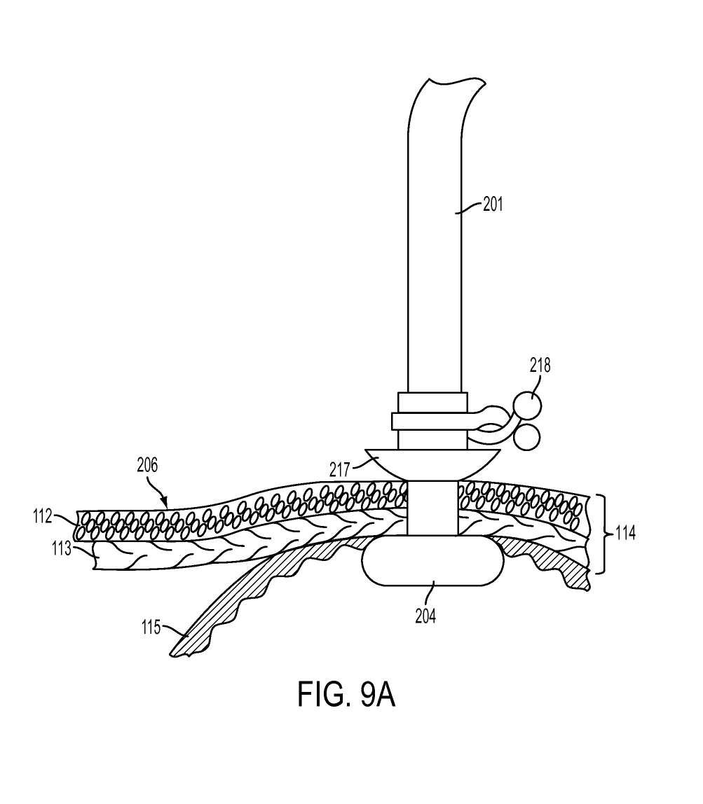

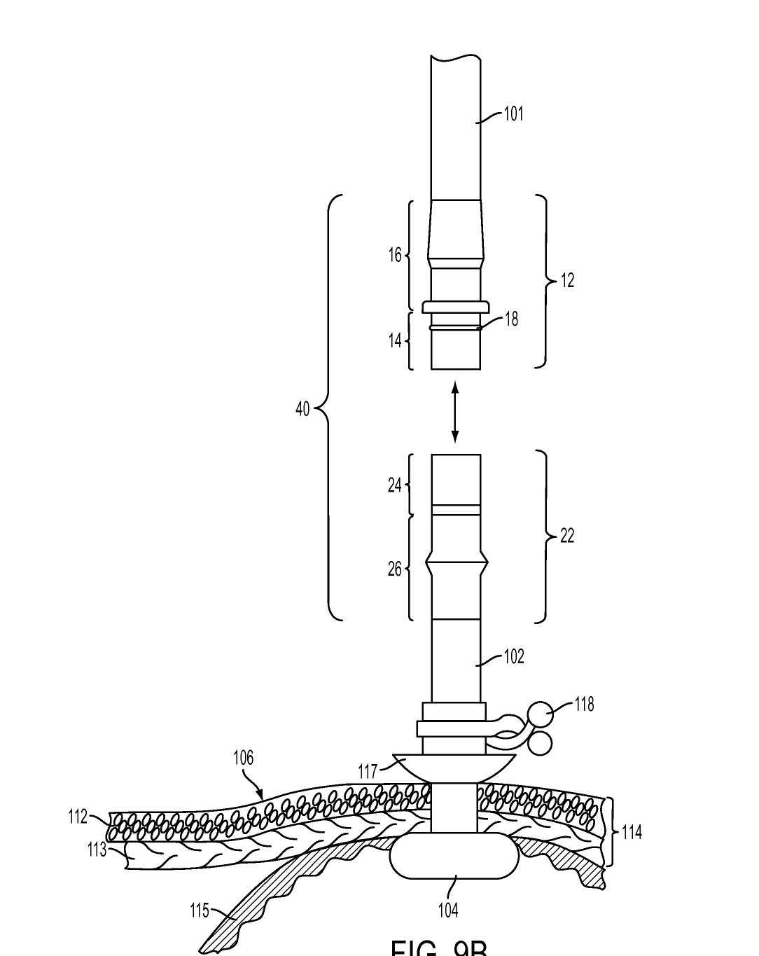

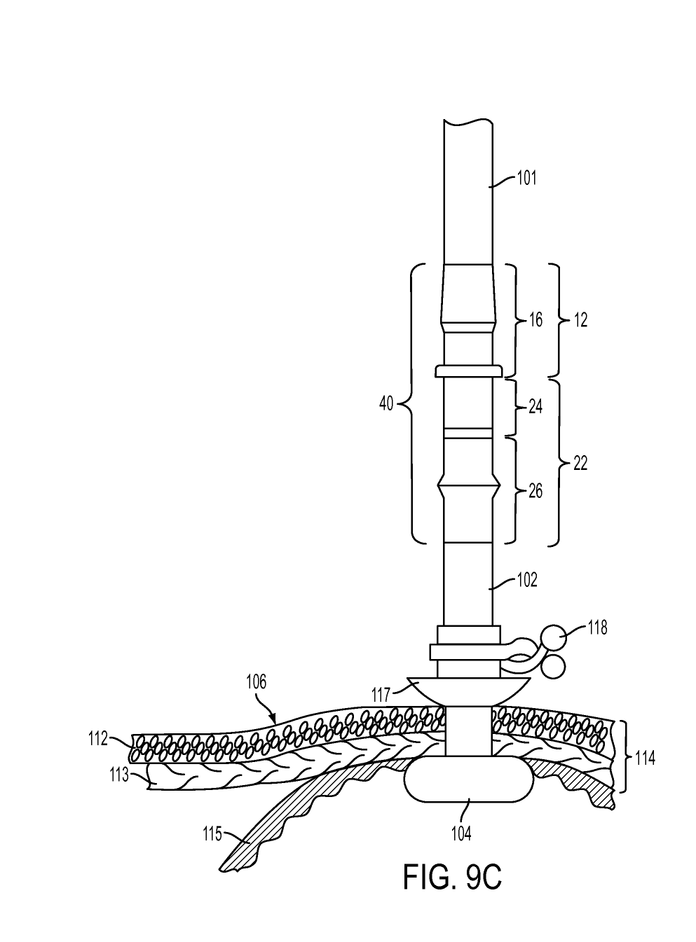

Referring now to FIG. 9(A), FIG. 9(A) provides an illustration of an elevation schematic view of a PEG tube 201. The PEG tube 201 is shown traversing the abdominal wall 114 (cutanious wall 112 and subcutaneous wall 113) and gastric wall 115 of the subject 206. Also shown is an external bolster 217 with a twist lock 218 located proximal to the abdominal wall 114. Also shown is an internal portion 204 inside the subject 206; whereby in the instant illustration the internal portion is disclosed as a bumper.

FIGS. 9(B)-(C) provide an elevation schematic view of an embodiment of the coupling 40 in a disconnected position and connected position, respectively.

FIG. 9(B) provides a perspective view of an embodiment of the coupling 40 in a disconnected position, which includes first connector 12 that is disconnected from the second connector 22. FIG. 9(B) provides an illustration of a PEG tube having a first channel 101 and second channel 102 with an embodiment of the coupling 40 installed therewith. Also shown is an external bolster 117 with a twist lock 118 located between the coupling 40 and the subject 106 proximal to the abdominal wall 114. The coupling 40 is installed externally near to the abdominal wall 114 (or in a location as desired or required) of the subject 106 and is comprised of a first connector 12 communicating with a first channel 101 and a second connector 22 communicating with a second channel 102. The second channel 102 passes through the subject's abdominal wall 114 and gastric wall 115 and has an internal portion 104 inside the subject 106; whereby in the instant illustration the internal portion is disclosed as a bumper. The first connector 12 includes a first interface member 14 with a protrusion 18 to be to be matted with the second interface member 24 of the second connector 22. It should be noted that the protrusion 18 of the first connector 12 may be mated with protrusion retention recess (not shown) of the second interface member 24.

FIG. 9(C) provides a perspective view of an embodiment of the coupling 40 shown in FIG. 9(B) wherein the coupling 40 is in a connected position having the first connector 12 connected with the second connector 22. It should be noted that first interface member 14 and its protrusion 18 is inside the second interface member 24 of the second connector 22 and therefore is not visible in the illustration.

Furthermore, it should be appreciated that various components of this device may be manufactured or formed from a multitude of materials that satisfy the working requirements of the invention. This includes, but is not limited to, plastics, polymers, composites, metals, alloys and any combination thereof. This also includes, but is not limited to, materials molded or otherwise formed in order to have changing properties in any fashion including, but not limited to, along their length or across their section. This change in properties may either be by section or continuous in nature. The materials selected for the device 1 may be selected based on ease of manufacturing, price, material properties such as density, strength, modulus of elasticity, electrical or thermal conductivity, and biological compatibility.

The device 1, any of its components or sub-components, or any portions thereof may be manufactured or formed from a multitude of materials that satisfy the working requirements of the invention. This includes, but is not limited to, plastics, polymers, composites, metals, alloys and any combination thereof. This also includes, but is not limited to, materials molded or otherwise formed in order to have changing properties in any fashion including along their length or across their section. This change in properties may either be sectioned or continuous in nature. The materials selected for the device 1 may be selected based on ease of manufacturing, price, material properties such as density, strength, modulus of elasticity, electrical or thermal conductivity, and biological compatibility.

The applicant contemplates within the context of this invention that it may be produced in any geometrical form with variable length, width, shape, size, or other dimensional variability to match the requirements of specific applications for use.

It should be appreciated that the device 1 may be manufactured in a variety of ways. Specifically, this includes forming, molding, casting, forging, or otherwise producing components, sub-components, or portions thereof. The device may be produced as an assembly of parts wherein those parts are attached in any manner, including but not limited to fusing, welding, friction fits, threaded connections, snap connections, adhesives, or any other method for connecting one component, sub-component, or any portion thereof to another component, sub-component or portion thereof. The device 1 may also be manufactured so as to combine different functional elements into a single, multi-function component that would take on the function of two otherwise separate components.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

It should be appreciated that various sizes, dimensions, contours, rigidity, shapes, flexibility and materials of any of the components or portions of components in the various embodiments discussed throughout may be varied and utilized as desired or required. Similarly, locations and alignments of the various components may vary as desired or required.

It should be appreciated that any of the components or modules referred to with regards to any of the present invention embodiments discussed herein, may be integrally or separately formed with one another. Further, redundant functions or structures of the components or modules may be implemented.

It should be appreciated that the device 1 and related components discussed herein may take on all shapes along the entire continual geometric spectrum of manipulation of x, y and z planes to provide and meet the anatomical and structural demands and operational requirements. Moreover, locations and alignments of the various components may vary as desired or required.

### EXAMPLES

Practice of an aspect of an embodiment (or embodiments) of the invention will be still more fully understood from the following examples and experimental results, which are presented herein for illustration only and should not be construed as limiting the invention in any way.

### Example and Experimental Set No. 1

Data Acquisition Study:

The Applicants' UVA Institutional Review Board (IRB)-approved trial (UVA-IRB#14526) was conducted on 60 patients to quantify the force required to intentionally remove a PEG from the stomach by gentle external traction. This data was used to determine the ideal force that an embodiment of the present invention device should disconnect. The force to remove the PEG was quantified by using a force measurement gauge (American Weigh® SR-20 Digital Hanging Scale 20×0.01 kg, and Extech Instruments Digital Force Gauge: Model 475044) to remove the PEGs out intentionally in clinic by external traction. This data allowed determination of the force (i.e., decoupling force) at which the device should separate in order to prevent the PEG from accidentally being pulled through the abdominal wall.

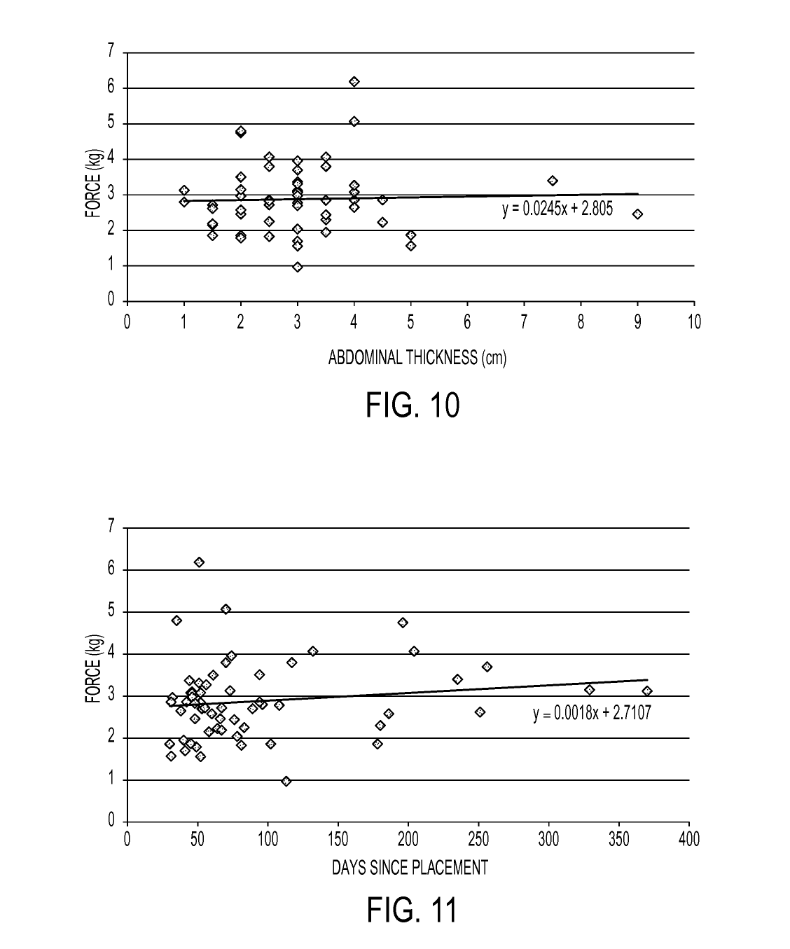

Referring to the graph of FIG. 10, data demonstrates the force to intentionally remove a PEG tube is not related to thickness of the abdominal wall. The trendline (y=0.0245x+2.805) is nearly horizontal (slope of zero) indicating no relationship between the abdominal thickness and force of dislodgement. Accordingly, as the device is removed when the internal bumper collapses to the diameter of the tube allowing easy passage through the abdominal wall, regardless of how far it must travel. This information reveals a single device, with a particular break force, will be applicable to all patients regardless of patient size or abdominal girth. The graph of FIG. 10 depicts the force (kg) of external traction for removal of PEG tubes through abdominal wall versus the thickness (cm) of the abdominal wall.

Referring to the graph of FIG. 11, the graph displays the days since placement or the “age” of the PEG tube. The data suggests the “age” of the tube is unrelated to the force required for removal (y=0.0018x+2.7107). The graph illustrates the force of external traction (kg) versus “age” (days) of the PEG tube.

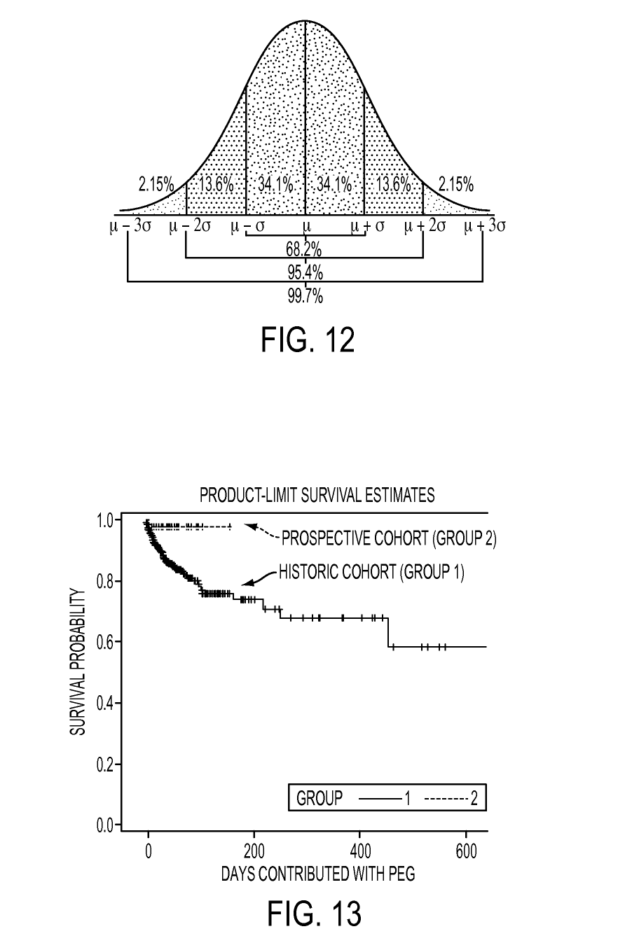

Turning to FIG. 12, the mean, ±the standard error of the mean, for the pull force from these 60 patients is 2.88 kg±0.12 with a standard deviation of 0.93 kg. The median pull force is 2.82 kg and the range is from 0.97 to 6.19 kg. Assuming a standard normal (Gaussian) distribution for this continuous data, one can determine the ideal break force. By taking two standard deviations above and below the mean one can predict to include 95.4% of all dislodgement data points. Accordingly, FIG. 12 graphically illustrates the Standard (Gaussian) distribution with mean (g) and standard deviations (a).

For example, two standard deviations (0.93 kg) below the mean (2.88 kg) is a value of 1.02 kg. This is the “force” at which the device will separate. If the device disconnects at approximately 1.0 kg of force one can predict it will prevent 97.6% of PEG dislodgements as calculated from the original data.

In summary, an aspect of an embodiment of the present invention PEG coupling device will prevent accidental removal of the PEG feeding tube by being a point of disconnection to prevent the PEG tube's internal bumper from collapsing and dislodging through the abdominal wall. External fraction placed anywhere along the length of the tubing will cause the two-piece device to separate, relieving all pressure from the internal bumper preventing its collapse and dislodgement. This device is applicable for all PEGs with a soft internal bumper and not those with an intragastric balloon.

A great benefit of various embodiments of the present invention device is that it does not require any pre-market modification of the PEG tubes and insertion kits. This can be added to the PEG tubing following placement and does not require a well-healed gastrocutaneous fistula tract (as is required for a replacement balloon gastrostomy tube). It is also designed to fit any 24-French PEG tubing and therefore is applicable regardless of PEG brand. Patient safety organizations have already become concerned with the restraining of patients, and more regulations have been put into place restricting the use of wrist restraints and hand mitts. With increasing regulations such as these, it is anticipated that there will be more frequent PEG dislodgements and an even greater need for a novel safety mechanism.

### Example and Experimental Set No. 2

An aspect of an embodiment of the present invention and related method is intended to prevent the accidental dislodgement of percutaneous endoscopic gastrostomy (PEG) tubes, as shown in FIG. 9(A). It is intended to be a point of disconnection when force (or external traction) is placed anywhere along the length of the tubing external to the patient.

The standard PEG tube provides direct access to the stomach and provides enteral nutrition via the long, flexible feeding tube with a soft internal bumper. Conveniently, the soft inner bumper of the PEG collapses and slides out through the tube tract, allowing the PEG to be removed with relatively minimal external traction. This feature allows easy, intentional removal in an outpatient, clinic setting without the need for an operation or sedation when the patient has recovered from their initial insult.

Consequently, due to this flexible inner bumper, a major complication is premature removal or accidental dislodgement, from which significant morbidity and mortality may occur. Secondary complications occur with premature removal as the gastrocutaneous fistula tract has not fully matured allowing the stomach to separate from the anterior abdominal wall and the open gastrostomy to leak gastric contents. Complications include peritonitis requiring laparotomy, abdominal wall necrotizing fasciitis, candida peritonitis, and hemoperitoneum following gastrostomy tube reinsertion. Accidental dislodgement rates are well published and have been reported as up to 12.8% when followed longitudinally for the lifetime of the originally placed PEG. See Rosenberger L H, Newhook T, Schirmer B, Sawyer R G. Late accidental dislodgement of the percutaneous endoscopic gastrostomy: an underestimated burden on patients and the healthcare system. Surgical Endoscopy. 2011; 25:3307-3311, of which is hereby incorporated by reference herein in its entirety.

While early dislodgements can be clinically devastating, late dislodgements after gastrocutanous fistula maturation are less detrimental, however, they may require expensive emergency department visits, surgical consultations, replacement tubes, and radiographic confirmation of position. See Rosenberger L H, Newhook T, Schirmer B, Sawyer R G., 2011 One aspect of an embodiment of the present invention is a solution to what has become the “Achilles Heel” of the PEG tube; accidental dislodgement.

Applicants conducted a study at the University of Virginia to assess the effectiveness of an embodiment of the present invention in a PEG tube as shown in FIGS. 9(B) and 9(C). The primary endpoint was the number of accidental dislodgements of the principally placed PEG tube. This study was designed as a prospective longitudinal cohort for comparison against a well-defined historical cohort. [See Rosenberger, L H, Newhook T, Schirmer B, Sawyer R G., 2011].

Following standard PEG placement, an embodiment of the invention was placed in close proximity to the patient's abdominal wall. Typically, PEG tubing was cut transversely approximately 2 cm above the top of the external bumper and the device was installed. This resulted in relatively short first channel and a longer second channel connected to the feeding apparatus.

A total of 53 patients completed the initial phase of the study, 1 resulting in accidental PEG dislodgement, 37 in intentional removal of the PEG, 0 with exchange of the original PEG for a replacement balloon gastrostomy, and 15 in death with the device in place. A Kaplan-Meier survival analysis was performed comparing the prospective cohort (PEG tubes with an embodiment of the present invention, N=53) with the historic cohort (PEG tubes without an embodiment of the invention, N=563). The analysis shows a clear and significant longer survival of a single PEG tube with an embodiment of the invention in place. The exemplary embodiment of invention is shown to prevent accidental dislodgement and allow any given PEG to remain in place longer than previously shown. Referring to the graph of FIG. 13, the log-rank test reveals a significant difference (p=0.043). The graph of FIG. 13 reveals a Kaplan-Meier analysis for current prospective cohort (Group 2) versus historic cohort (Group 1).

The top line represents the prospective cohort with invention in place and the bottom line represents the historical cohort without invention in place

### Example and Experimental Set No. 3

It should be appreciated that as an embodiment of the invention has demonstrated effectiveness in preventing the accidental dislodgment of PEG tubes, further embodiments should be equally able to prevent accidental dislodgment of other tubes and channels that have an internal portion inside a subject. Such other embodiments of tubes or channels, and applications include, but are in no way limited to:

Foley Catheter—where the internal portion of the catheter is positioned in the bladder for gravity drainage. An external force on the tubing can cause disruption of the internal portion of the catheter. An embodiment of the invention could be installed in-line, a short distance from the body and would separate when a tractive force was applied that would typically disrupt the internal portion of the catheter.

Intraventricular drain—where the internal portion of the drain channel is positioned inside the ventricles of the brain and where an external force on the external portion of the channel can cause disruption of the internal portion of the channel. An embodiment of the invention could be installed in-line, a short distance from the body and would separate when a tractive force was applied that would typically disrupt the internal portion of the channel.

Chest tube—where the internal portion of the chest tube sits inside the thoracic cavity between the lungs and the chest wall. The chest tube is designed to drain air, fluid, or blood from the thoracic cavity and external force on the tube may cause disruption of the internal position of the tube. An embodiment of the invention could be installed in-line, a short distance from the body and would separate when a tractive force was applied that would typically disrupt the internal portion of the tube.

Nephrostomy tubes—where an internal “pigtail end” of the catheter sits in the renal pelvis to drain urine from the kidney. This pigtail end may be disrupted when an external force is applied to the external portion of the catheter. An embodiment of the invention could be installed in-line, a short distance from the body and would separate when a tractive force was applied that would typically disrupt the pigtail end of the catheter.

Percutaneous transhepatic cholangiography (“PTC”) tube—where an internal channel is placed into the biliary tree to allow bile drainage. Such channel can be disrupted when a force is applied to the external portion of the channel. An embodiment of the invention could be installed in-line, a short distance from the body and would separate when a tractive force was applied that would typically disrupt the internal portion of the channel.

Pigtail catheters for abscess drainage—where an internal “pigtail end” of the catheter sits in any number of intra-abdominal locations to drain fluid from an abscess. This pigtail end may be disrupted when an external force is applied to the external portion of the catheter. An embodiment of the invention could be installed in-line, a short distance from the body and would separate when a tractive force was applied that would typically disrupt the pigtail end of the catheter.

Generally, any channel that has an internal portion inside a subject is subject to disruption when a force is applied to the external portion of the channel. An embodiment of this invention may be placed in-line with the channel to prevent the disruption of the internal portion of the channel when an applicable force is applied beyond the invented device. Similarly, an embodiment of the invention may be implemented with an optimal or desired decoupling force according to the teachings, techniques, structures, components and principles set forth in this disclosure regarding the various embodiments or aspects of the present invention.

### ADDITIONAL EXAMPLES

### Example 1

An assembly device for use in a medical environment to be used with a first channel and a second channel, wherein the first channel has an internal portion located at an intended position inside a subject. The device may comprise: a first connector comprising a first interface member and a channel communication section; a second connector comprising a second interface member and a channel communication section; the first interface member of the first connector and the second interface member of the second connector configured to join together to form a coupling configured to allow fluid to flow between the first channel and the second channel, the coupling having a decoupling force; wherein the coupling is joined to the first channel and the second channel at the communication section of the first connector and the communication section of the second connector; and wherein the decoupling force is the force required to separate the first connector and the second connector from one another to allow the internal portion to maintain its intended position in the subject.

### Example 2

The device of example 1, wherein the first connector comprises a male connector and the first interface member comprises an insert section.

### Example 3

The device of example 2, wherein the second connector comprises a female connector and the second interface member comprises a receiving section, whereby the coupling is provided by the receiving section receiving the insert section to provide the joining together.

### Example 4

The device of example 1 (as well as subject matter of one or more of any combination of examples 2-3), wherein the first channel and/or second channel comprises: a tube, a conduit, a port or any combination thereof.

### Example 5

The device of example 4 (as well as subject matter of one or more of any combination of examples 2-3), wherein the tube comprises a percutaneous endoscopic gastrostomy (PEG).

### Example 6

The device of example 1 (as well as subject matter of one or more of any combination of examples 2-5), wherein the decoupling force is between 0.5 kilogram-force and 2.5 kilograms-force.

### Example 7

The device of example 1 (as well as subject matter of one or more of any combination of examples 2-6), wherein the decoupling force is between 1.1 kilograms-force and 1.3 kilograms-force.

### Example 8

The device of example 1 (as well as subject matter of one or more of any combination of examples 2-7), wherein the channel communication section 16 on the first connector is configured to have an attachment means for attaching to the first channel and/or the second channel.

### Example 9

The device of example 1 (as well as subject matter of one or more of any combination of examples 2-8), wherein the channel communication section 26 on the second connector is configured to have an attachment means for attaching to the first channel and/or the second channel.

### Example 10

The assembly of example 1 (as well as subject matter of one or more of any combination of examples 2-9), wherein the joining of the first interface member of the first connector and the second interface member of the second connector provides faying surfaces, wherein the faying surfaces forms a leak inhibiting seal.

### Example 11

The device of example 1 (as well as subject matter of one or more of any combination of examples 2-10), wherein the first interface member of the first connector includes one or more circumferentially oriented protrusions.

### Example 12

The device of example 7 (as well as subject matter of one or more of any combination of examples 1-6 or 8-11), wherein the one or more circumferentially oriented protrusions are a continuous segment.

### Example 13

The device of example 7 (as well as subject matter of one or more of any combination of examples 1-6 or 8-12), where the one or more circumferentially oriented protrusions are discontinuous segments.

### Example 14

The device of example 7 (as well as subject matter of one or more of any combination of examples 1-6 or 8-13), wherein the second interface member of second connector includes one or more protrusion retention recesses.

### Example 15

The device of example 1 (as well as subject matter of one or more of any combination of examples 2-14), wherein the decoupling force is the force required to separate the first connector and the second connector from one another to allow the internal portion to maintain its intended position in the subject and to allow the separated the first connector and the second connector to remain joined to their the respective channels.

### Example 16

An assembly device for use in a medical environment to be used with a first channel and a second channel, wherein the first channel has an internal portion located at an intended position inside a subject. The device may comprise: a first connector; a second connector; the first connector and the second connector configured to join together to form a coupling configured to allow fluid to flow between the first channel and the second channel, the coupling having a decoupling force; wherein the coupling is joined between the first channel and the second channel; and wherein the decoupling force is the force required to separate the first connector and the second connector from one another to allow the internal portion to maintain its intended position in the subject. Moreover, the system may include subject matter of one or more of any combination of examples 1-15.

### Example 17

The device of example 16 (as well as subject matter of one or more of any combination of examples 1-15), wherein the first channel and/or second channel comprises: a tube, a conduit, a port or any combination thereof.

### Example 18

The device of example 17 (as well as subject matter of one or more of any combination of examples 1-15), wherein the tube comprises a percutaneous endoscopic gastrostomy (PEG).

### Example 19

The device of example 16 (as well as subject matter of one or more of any combination of examples 1-15 or 17-18), wherein the decoupling force is between 0.5 kilogram-force and 2.5 kilograms-force.

### Example 20

The device of example 16 (as well as subject matter of one or more of any combination of examples 1-15 or 17-19), wherein the decoupling force is between 1.1 kilograms-force and 1.3 kilograms-force.

### Example 21

The device of example 16 (as well as subject matter of one or more of any combination of examples 1-15 or 17-20), wherein the first connector is attached to the first channel using an attachment means.

### Example 22

The device of example 16 (as well as subject matter of one or more of any combination of examples 1-15 or 17-21), wherein the first connector is attached to the first channel by way of at least one of the following connectors: male to female friction connector, magnetic connector, connector that uses adhesive, friction connection, ridge/valley connection, snap/click connection, O-ring connection, and screw/twist type mechanism connection.

### Example 23

The device of example 16 (as well as subject matter of one or more of any combination of examples 1-15 or 17-22), wherein the second connector is attached to the second channel using an attachment means.

### Example 24

The device of example 16 (as well as subject matter of one or more of any combination of examples 1-15 or 17-23), wherein the second connector is attached to the second channel by way of at least one of the following connectors: male to female friction connector, magnetic connector, connector that uses adhesive, friction connection, ridge/valley connection, snap/click connection, O-ring connection, and screw/twist type mechanism connection.

### Example 25

The device of example 16 (as well as subject matter of one or more of any combination of examples 1-15 or 17-24), wherein the first connector is joined to the second connector forming the coupling, wherein the coupling comprises at least one of the following: male to female friction connector, magnetic connector, connector that use adhesive, friction connection, ridge/valley connection, snap/click connection, O-ring connection, and screw/twist type mechanisms connection.

### Example 26

The device of example 16 (as well as subject matter of one or more of any combination of examples 1-15 or 17-25), wherein the decoupling force is the force required to separate the first connector and the second connector from one another to allow the internal portion to maintain its intended position in the subject and to allow the first connector to remain joined to the first channel and the second connector to remain connected to the second channel.

### Example 27

A system for use in a medical environment, wherein the system comprises: a first channel; a second channel, wherein the first channel has an internal portion located at an intended position inside a subject; and an assembly device. The assembly device may comprise: a first connector comprising a first interface member and a channel communication section; a second connector comprising a second interface member and a channel communication section; the first interface member of the first connector and the second interface member of the second connector configured to join together to form a coupling configured to allow fluid to flow between the first channel and the second channel, the coupling having a decoupling force; wherein the coupling is joined to the first channel and the second channel at the communication section of the first connector and the communication section of the second connector; and wherein the decoupling force is the force required to separate the first connector and the second connector from one another to allow the internal portion to maintain its intended position in the subject. Moreover, the system may include subject matter of one or more of any combination of examples 1-26.

### Example 28

A system for use in a medical environment, wherein the system comprises: a first channel; a second channel, wherein the first channel has an internal portion located at an intended position inside a subject; and an assembly device. The assembly device may comprise: a first connector; a second connector; the first connector and the second connector configured to join together to form a coupling configured to allow fluid to flow between the first channel and the second channel, the coupling having a decoupling force; wherein the coupling is joined between the first channel and the second channel; and wherein the decoupling force is the force required to separate the first connector and the second connector from one another to allow the internal portion to maintain its intended position in the subject. Moreover, the system may include subject matter of one or more of any combination of examples 1-27.

### Example 29

A method used in a medical environment to be used with a first channel and a second channel, wherein the first channel has an internal portion located at an intended position inside the subject. The method may comprise: providing a first connector; providing a second connector; joining the first connector and the second connector to form a coupling to allow fluid to flow between the first channel and the second channel, the coupling having a decoupling force; wherein the coupling is joined to the first channel and the second channel; and wherein the decoupling force is the force required to separate the first connector and the second connector from one another to allow the internal portion to maintain its intended position. Moreover, the system may include subject matter of one or more of any combination of examples 1-28.

### Example 30

The device of example 29 (as well as subject matter of one or more of any combination of examples 1-28), wherein the first connector comprises a male connector and the first interface member comprises an insert section.

### Example 31

The device of example 30 (as well as subject matter of one or more of any combination of examples 1-28), wherein the second connector comprises a female connector and the second interface member comprises a receiving section, whereby the coupling is provided by the receiving section receiving the insert section to provide the joining together.

### Example 32

The method of example 29 (as well as subject matter of one or more of any combination of examples 1-28 or 30-31), wherein the first channel and/or second channel comprises: a tube, a conduit, a port or any combination thereof.

### Example 33

The method of example 32 (as well as subject matter of one or more of any combination of examples 1-28 or 30-31), wherein the tube comprises a percutaneous endoscopic gastrostomy (PEG).

### Example 34

The method of example 29 (as well as subject matter of one or more of any combination of examples 1-28 or 30-33), wherein the decoupling force is between 0.5 kilogram-force and 2.5 kilograms-force.

### Example 35

The method of example 29 (as well as subject matter of one or more of any combination of examples 1-28 or 30-34), wherein the decoupling force is between 1.1 kilograms-force and 1.3 kilograms-force.

### Example 36

The method of example 29 (as well as subject matter of one or more of any combination of examples 1-28 or 30-35), wherein joining of the first interface member of the first connector and the second interface member of the second connector provides faying surfaces, wherein the faying surfaces forms a leak inhibiting seal.

### Example 37

The method of example 29 (as well as subject matter of one or more of any combination of examples 1-28 or 30-36), wherein the decoupling force is the force required to separate the first connector and the second connector from one another to allow the internal portion to maintain its intended position in the subject and to allow the first connector to remain joined to the first channel and the second connector to remain connected to the second channel.

### Example 38

A method used in a medical environment, whereby the method comprises: providing a first channel; providing a second channel, wherein the first channel has an internal portion located at an intended position inside the subject; providing a first connector; providing a second connector; joining the first connector and the second connector to form a coupling to allow fluid to flow between the first channel and the second channel, the coupling having a decoupling force; wherein the coupling is joined to the first channel and the second channel; and wherein the decoupling force is the force required to separate the first connector and the second connector from one another to allow the internal portion to maintain its intended position. Moreover, the system may include subject matter of one or more of any combination of examples 1-37.

### Example 39

A method of manufacturing any of the devices or systems (or portions thereof) provided in one or more of any combination of examples 1-28; by implementing, but not limited thereto, fabrication techniques and material selection known to one skilled in art.

The devices, systems, compositions, and methods of various embodiments of the invention disclosed herein may utilize aspects disclosed in the following references, applications, publications and patents and which are hereby incorporated by reference herein in their entirety (and which are not admitted to be prior art with respect to the present invention by inclusion in this section):
1. Gauderer M W. Percutaneous Endoscopic Gastrostomy-20 Years Later: A Historical Perspective. Journal of Pediatric Surgery. 2001; 36(1): 217-219.
2. Ponsky J L, Gauderer M W. Percutaneous Endoscopic Gastrostomy: a Non-operative technique for feeding gastrostomy. Gastrointestinal Endoscopy. 1981; 27(1): 9-11.
3. University of Virginia, Clinical Data Repository. Project: “5-year PEG data”. Search criteria: CPT code 43246 from Jan. 1, 2005-Dec. 31, 2009. Search completed: Dec. 17, 2010.
4. Schapiro G D, Edmundowicz S A. Complications of percutaneous endoscopic gastrostomy. Gastrointest Endosc Clin N Am. 1996; 6:409-422.
5. Marshall J B, Bodnarchuk G, Barthel J S. Early accidental dislodgement of PEG tubes. J Clin Gastroenterol. 1994; 18:210-212.
6. Maclean A A, Miller G, Bamboat Z M, Hiotis K. Abdominal wall necrotizing fasciitis from dislodged percutaneous endoscopic gastrostomy tubes: a case series. Am Surg. 2004; 70:827-831.
7. Sridhar A V, Nichani S, Luyt D, Nour S. Candida peritonitis: a rare complication following early dislodgement of percutaneous endoscopic gastrostomy tube. J Paediatr Child Health. 2006; 42:145-146.
8. Tan Y M, Abdullah M, Goh K L. Hemoperitoneum after accidental dislodgement and subsequent replacement of PEG tube. Gastrointest Endosc. 2001; 53:671-673.
9. Chowdhury, M A. Batey, R. Complications and outcome of percutaneous endoscopic gastrostomy indifferent patient groups. Journal of Gastroenterology and Hepatology. 1996; 11:835-839.
10. Rosenberger L H, Newhook T, Schirmer B, Sawyer R G. Late accidental dislodgement of the percutaneous endoscopic gastrostomy: an underestimated burden on patients and the healthcare system. Surgical Endoscopy. 2011; 25:3307-3311.
11. Boston Scientific Initial Placement PEG Print Materials. Accessed at: <http://www.genesyshtasystem.com/Device.bsci?page=HCP_Overview&navRelId=1 000.1003&method=DevDetailHCP&id=10119432&pageDisclaimer=Disclaimer.ProductPage>
12. Cook Medical Incorporated. Product materials. Accessed at: <http://www.cookmedical.com/esc/content/mmedia/18731_{—}0710.pdf>
13. Covidien Product Materials. Purple: the new standard for enteral nutrition safety. Accessed at: <http://www.covidien.com/imageServer.aspx?contentID=13294&contenttype=application/pdf>
14. American Society for Gastrointestinal Endoscopy (ASGE) Technology Committee. Technology Status Evaluation Report: Enteral nutrition access devices. Gastrointestinal Endoscopy. 2010; 72:236-248.
15. U.S. Patent Application Publication No. US 2009/0204097 A1, Knight, T., “Enteral Feeding Safety Reservoir and System”, Aug. 13, 2009.
16. U.S. Patent Application Publication No. US 2008/0195047 A1, Price, M., “Enteral Feeding Systems, Devices and Methods”, Aug. 14, 2008.
17. U.S. Pat. No. 5,549,657, Stern, et al., “Low Profile Adaptor for Gastrostomy Feeding Tube”, Aug. 27, 1996.
18. U.S. Patent Application Publication No. US 2009/0216186 A1, Nath, I., “Feeding Tube”, Aug. 27, 2009.
19. U.S. Pat. No. 6,419,670 B1, Dikeman, W. Cary, “Gastrostomy Tube Set”, Jul. 16, 2002.
20. U.S. Pat. No. 7,481,796 B2, Nishtala, et al., “Low Profile Retention System”, Jan. 27, 2009.
21. U.S. Patent Application Publication No. US 2008/0125719 A1, Takuma, N., “Gastro-Tube Using a Shape-Retentive Alloy and Method of Use Thereof”, May 29, 2008.
22. U.S. Pat. No. 5,391,159, Hirsch, et al., “Gastrostomy Tube With Improved Internal Retaining Member”, Feb. 21, 1995.
23. Rosenberger L H, Newhook T, Schirmer B, Sawyer R G. Late accidental dislodgement of the percutaneous endoscopic gastrostomy: an underestimated burden on patients and the healthcare system. Surgical Endoscopy. 2011; 25:3307-3311.
24. American Society for Gastrointestinal Endoscopy (ASGE) Technology Committee. Technology Status Evaluation Report: Enteral nutrition access devices. Gastrointestinal Endoscopy. 2010; 72:236-248.
25. PCT Publication No. WO 2012/088463 A1, to Williams et al., “Breakaway Coupling Assembly,” Jun. 28, 2012 (PCT Application PCT/US2011/066951, filed Dec. 22, 2011).

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

We claim:

1. An assembly device for use in a medical environment to be used with a first channel and a second channel, wherein the first channel has an internal portion located at an intended position inside a subject, said device comprising:
a first connector comprising a first interface member and a channel communication section;
a second connector comprising a second interface member and a channel communication section;
said first interface member of said first connector and said second interface member of said second connector configured to join together to form a coupling configured to allow fluid to flow between the first channel and the second channel, said coupling having a decoupling force;
wherein said coupling is joined to the first channel and the second channel at said communication section of said first connector and said communication section of said second connector; and
wherein said decoupling force is the force required to separate said first connector and the second connector from one another to allow the internal portion to maintain its intended position in the subject.

2. The device of claim 1, wherein said first connector comprises a male connector and said first interface member comprises an insert section.

3. The device of claim 2, wherein said second connector comprises a female connector and said second interface member comprises a receiving section, whereby the coupling is provided by said receiving section receiving said insert section to provide the joining together.

4. The device of claim 1, wherein said first channel and/or second channel comprises: a tube, a conduit, a port or any combination thereof.

5. The device of claim 4, wherein said tube comprises a percutaneous endoscopic gastrostomy (PEG).

6. The device of claim 1, wherein the decoupling force is between 0.5 kilogram-force and 2.5 kilograms-force.

7. The device of claim 1, wherein the decoupling force is between 1.1 kilograms-force and 1.3 kilograms-force.

8. The device of claim 1, wherein said channel communication section 16 on said first connector is configured to have an attachment means for attaching to the first channel and/or the second channel.

9. The device of claim 1, wherein said channel communication section 26 on said second connector is configured to have an attachment means for attaching to the first channel and/or the second channel.

10. The assembly of claim 1, wherein said joining of said first interface member of said first connector and said second interface member of said second connector provides faying surfaces, wherein said faying surfaces forms a leak inhibiting seal.

11. The device of claim 1, wherein said first interface member of said first connector includes one or more circumferentially oriented protrusions.

12. The device of claim 7, wherein said one or more circumferentially oriented protrusions are a continuous segment.

13. The device of claim 7, where said one or more circumferentially oriented protrusions are discontinuous segments.

14. The device of claim 7, wherein said second interface member of second connector includes one or more protrusion retention recesses.

15. The device of claim 1, wherein said decoupling force is the force required to separate said first connector and said second connector from one another to allow the internal portion to maintain its intended position in the subject and to allow the separated said first connector and said second connector to remain joined to their said respective channels.

16. An assembly device for use in a medical environment to be used with a first channel and a second channel, wherein the first channel has an internal portion located at an intended position inside a subject, said device comprising:
a first connector;
a second connector;
said first connector and said second connector configured to join together to form a coupling configured to allow fluid to flow between the first channel and the second channel, said coupling having a decoupling force;
wherein said coupling is joined between the first channel and the second channel; and
wherein said decoupling force is the force required to separate said first connector and the second connector from one another to allow the internal portion to maintain its intended position in the subject.

17. The device of claim 16, wherein said first channel and/or second channel comprises: a tube, a conduit, a port or any combination thereof.

18. The device of claim 17, wherein said tube comprises a percutaneous endoscopic gastrostomy (PEG).

19. The device of claim 16, wherein the decoupling force is between 0.5 kilogram-force and 2.5 kilograms-force.

20. The device of claim 16, wherein the decoupling force is between 1.1 kilograms-force and 1.3 kilograms-force.

21. The device of claim 16, wherein said first connector is attached to the first channel using an attachment means.

22. The device of claim 16, wherein said first connector is attached to the first channel by way of at least one of the following connectors: male to female friction connector, magnetic connector, connector that uses adhesive, friction connection, ridge/valley connection, snap/click connection, O-ring connection, and screw/twist type mechanism connection.

23. The device of claim 16, wherein said second connector is attached to the second channel using an attachment means.

24. The device of claim 16, wherein said second connector is attached to the second channel by way of at least one of the following connectors: male to female friction connector, magnetic connector, connector that uses adhesive, friction connection, ridge/valley connection, snap/click connection, O-ring connection, and screw/twist type mechanism connection.

25. The device of claim 16, wherein said first connector is joined to said second connector forming said coupling, wherein said coupling comprises at least one of the following: male to female friction connector, magnetic connector, connector that use adhesive, friction connection, ridge/valley connection, snap/click connection, O-ring connection, and screw/twist type mechanisms connection.

26. The device of claim 16, wherein said decoupling force is the force required to separate said first connector and the second connector from one another to allow the internal portion to maintain its intended position in the subject and to allow said first connector to remain joined to said first channel and said second connector to remain connected to said second channel.

27. A system for use in a medical environment, wherein said system comprises:
a first channel;
a second channel, wherein the first channel has an internal portion located at an intended position inside a subject; and
an assembly device, wherein said assembly device comprises:
a first connector comprising a first interface member and a channel communication section;
a second connector comprising a second interface member and a channel communication section;
said first interface member of said first connector and said second interface member of said second connector configured to join together to form a coupling configured to allow fluid to flow between said first channel and said second channel, said coupling having a decoupling force;
wherein said coupling is joined to said first channel and said second channel at said communication section of said first connector and said communication section of said second connector; and
wherein said decoupling force is the force required to separate said first connector and the second connector from one another to allow the internal portion to maintain its intended position in the subject.

28. A system for use in a medical environment, wherein said system comprises:
a first channel;
a second channel, wherein the first channel has an internal portion located at an intended position inside a subject; and
an assembly device, wherein said assembly device comprises:
a first connector;
a second connector;
said first connector and said second connector configured to join together to form a coupling configured to allow fluid to flow between said first channel and said second channel, said coupling having a decoupling force;
wherein said coupling is joined between said first channel and said second channel; and
wherein said decoupling force is the force required to separate said first connector and the second connector from one another to allow the internal portion to maintain its intended position in the subject.

29. A method used in a medical environment to be used with a first channel and a second channel, wherein said first channel has an internal portion located at an intended position inside the subject, said method comprising:
providing a first connector;
providing a second connector;
joining said first connector and said second connector to form a coupling to allow fluid to flow between the first channel and the second channel, said coupling having a decoupling force;
wherein said coupling is joined to the first channel and the second channel; and
wherein said decoupling force is the force required to separate said first connector and said second connector from one another to allow the internal portion to maintain its intended position.

30. The device of claim 29, wherein said first connector comprises a male connector and said first interface member comprises an insert section.

31. The device of claim 30, wherein said second connector comprises a female connector and said second interface member comprises a receiving section, whereby the coupling is provided by said receiving section receiving said insert section to provide the joining together.

32. The method of claim 29, wherein said first channel and/or second channel comprises: a tube, a conduit, a port or any combination thereof.

33. The method of claim 32, wherein said tube comprises a percutaneous endoscopic gastrostomy (PEG).

34. The method of claim 29, wherein the decoupling force is between 0.5 kilogram-force and 2.5 kilograms-force.

35. The method of claim 29, wherein the decoupling force is between 1.1 kilograms-force and 1.3 kilograms-force.

36. The method of claim 29, wherein joining of said first interface member of said first connector and said second interface member of said second connector provides faying surfaces, wherein said faying surfaces forms a leak inhibiting seal.

37. The method of claim 29, wherein said decoupling force is the force required to separate said first connector and the second connector from one another to allow the internal portion to maintain its intended position in the subject and to allow said first connector to remain joined to said first channel and said second connector to remain connected to said second channel.

38. A method used in a medical environment, said method comprising:
providing a first channel;
providing a second channel, wherein said first channel has an internal portion located at an intended position inside the subject;
providing a first connector;
providing a second connector;
joining said first connector and said second connector to form a coupling to allow fluid to flow between said first channel and said second channel, said coupling having a decoupling force;
wherein said coupling is joined to said first channel and said second channel; and
wherein said decoupling force is the force required to separate said first connector and said second connector from one another to allow the internal portion to maintain its intended position.
